(19) United States
(12) Patent Application Publication
(10) Pub. No.: US 2003/0161872 A1
(43) Pub. Date: Aug. 28, 2003

(54) Capsule for holding liquid-containing compositions and method for making the same

(76) Inventors: Gan-Lin Chen, Taipei (TW); Wei-Hua Hao, Taipei (TW)

Correspondence Address: VENABLE, BAETJER, HOWARD AND CIVILETTI, LLP, P.O. BOX 34385, WASHINGTON, DC, 20043-9998, US

(21) Appl. No.: 10/035,242

(22) Filed: Jan. 4, 2002

(51) Int. Cl. A61K 9/48

(52) U.S. Cl. 424/452

(57) ABSTRACT

The present invention provides a capsule which comprises (1) a capsule shell, and (2) a liquid core composition. The capsule shell comprises a pH-dependent polymer and optionally a plasticizer. The liquid core composition contains liquid up to 70% by volume. The pH of the liquid core composition is adjusted to or at a pH in which the pH-dependent polymer is insoluble. The liquid core composition is preferably a decoction or condensate of the decoction containing herbal extract. The present invention further provides methods for making the capsule.

### FIELD OF THE INVENTION

The present invention relates to a capsule which is characterized by its capacity to hold liquid-containing compositions wherein the liquid content can be up to 70% by volume. The capsule contains (1) a capsule shell, and (2) a liquid core composition. The capsule shell comprises a pH-dependent polymer, preferably methacrylic acid/ester copolymer, and optionally a plasticizer. The liquid core composition is adjusted to or at a pH in which the pH-dependent polymer is insoluble. The preferred liquid core composition is a decoction or a condensate of the decoction containing herbal extract. The capsule is preferred to be used for oral administration of liquid pharmaceutical compositions. The present invention also relates to methods for making the capsule.

### DESCRIPTION OF THE RELATED ART

Capsules for delivering a composition having high content of water or other moisture are confronted with problems of breakage or leakage due to the pressure built up by the water within the capsules. This becomes especially serious when the capsule is made of materials that are water-soluble, because the water content inside the capsule would dissolve the capsule shell.

Unfortunately, there are some compositions that are composed of ingredients which are desirably provided in high water or moisture content. For example, some herbal compositions are better prepared by decoction in water or other aqueous solutions. These herbal compositions, when condensed, still contain a significant amount of water or other aqueous solutions. In fact, most of the condensates of herbal compositions cannot be condensed to less than 20% by volume of water or other aqueous solutions. Heating, reduced-pressure dehydration, or addition of excipient may be used for removing the water. However, such methods not only are cumbersome and expensive, but also run the risks of losing the effectiveness of the herbal compositions.

To solve the problem, excess grease or oily substance has been reported to add to and mixed with liquid-containing pharmaceutical compositions for up to 66% by weight to insulate the water in the compositions from contacting the capsule shell. However, the excess grease in the capsule fills up the space in the capsule so that less amount of the composition is permitted to be included in the capsule so that a patient is required to take more capsules in order to obtain sufficient dosage for the pharmaceutical composition. In addition, some patients are allergic to grease and may develop discomfort or sickness due to the intake of excess grease.

A capsule for delivering a composition containing high water or moisture content has not been disclosed. The closest reference is U.S. Pat. No. 6,238,696 (the '696 patent), which discloses a process for providing a capsule containing vegetable gelatin, hydroxypropyl methylcellulose (HPMC), or other cellulose derivative. This capsule can hold non-lipid, liquid form of herbal extracts. However, the herbal extracts that are used in the '696 patent are prepared in such a way that a glycerin-based liquid or semi-solid herbal extract with a moisture content of less than 10% by weight, and preferably less than 5% by weight, is produced. Therefore, the capsule disclosed in the '696 patent does not present a challenge for holding an herbal extract containing more than 10% of water.

In the invention to be presented in the following section, a capsule which is particularly designed for holding high content of liquid is described. The capsule is especially suitable for delivering herbal extracts where the water content in the extracts are difficult to remove.

### SUMMARY OF THE INVENTION

The present invention provides a capsule which contains a capsule shell which encapsulates a liquid core composition. The capsule shell can be a hard or soft capsule shell, preferably a soft capsule shell. The capsule shell comprises a pH-dependent polymer and optionally a plasticizer. The preferred amount of the plasticizer is about 0-40%, more preferably 0.01%-10%. The liquid core composition contains up to 70%, preferably 50%, by volume of liquid. The preferred liquid is water. The preferred liquid core composition is an herbal extract, either a decoction or a condensate of the decoction. The herbal extract can be for pharmaceutical use or as dietary supplement. The pH of the liquid core composition is adjusted to a pH in which the pH-dependent polymer is insoluble.

Examples of acid insoluble polymer include, but are limited to, (1) a cellulose-based polymer, such as cellulose acetates, cellulose acetate phthalate, cellulose acetate succinate, cellulose acetate trimellitate, hydroxypropyl methylcellulose phthalate, hydroxypropyl methylcellulose acetate succinate, and carboxymethyl ethyl cellulose, (2) starch, sugar, polyols derivatives: starch acetate phthalate and amylose acetate phthalate, (3) polyvinyl derivatives: polyvinyl alcohol phthalate, polyvinyl acetate phthalate, polyvinyl butyrate phthalate, styrenemaleic acid copolymer, (4) acrylate polymers: polyacrylate, polymethylacrylate, poly(acrylate-methylacrylate), poly(methacrylate-methylmethacrylate), and poly(ethylacrylate-methylmethacrylate). In addition, 2-methyl-5-vinyl-pyridine-methyacrylate-methyacrylic acid is insoluble between pH 4 and 7.4. Shellac (which is a resinous excretion of the insect Laccifer (Tachardia) lacca Kerr, order Homoptera, family Coccidae) is acid insoluble. Zein (which is a prolamin obtainable from corn [molecular weight about 40,000] and which does not contain tryptophan or lysine) is insoluble at pH equal to or less than 11.5.

Examples of alkaline insoluble polymer include, poly [butyl methacrylate, (2-dimethylaminoethyl) methacrylate, methyl methacrylate], 2-methyl-5-vinyl-pyridine-methyacrylate-methyacrylic acid, and polyvinylacetal diethylamminoacetate.

Examples of the plasticizer which can be used together with the pH-dependent polymer include, but are not limited to, glycerin, propylene glycol, polyethylene glycol (PEG 200-6000), diethyl phthalate, dibutyl phthalate, dibutyl sebacate, triethyl citrate, acetyltriethyl citrate, acetyltributyl citrate, tributyl citrate, triacetyl glycerin, castor oil, acetylated monoglyceride, and coconut oil. The capsule shell composition can further contain gelatin. Gelatin is derived from collagen, which is an animal by-product. Collagen is found in animal bones, animal skins, and white animal connective tissues. When the collagen-containing animal tissue is boiled in water, it leaves behind gelatin, which is a colorless or pale yellow protein.

In addition, the inner side of the capsule shell can be deposited with oily substances to further insulate the capsule shell from the liquid core composition. The oily substances can be any combinations of vegetable oil, mineral oil, lanolin, emulsified wax, esterified wax, microcrystalline wax, white wax, yellow wax, lubricant lecithin, fatty oil, fatty acid, fish oil, liver oil, whale fatty oil, and polymethyleneglycol.

When the pH-dependent polymer is insoluble in acidic pH, the pH of the liquid core composition is adjusted to or at an acidic pH. The preferred pH-dependent polymer that is insoluble in acidic pH include, but are not limited to, polyacrylate, polymethylacrylate, poly(acrylate-methylacrylate), poly(methacrylate-methylmethacrylate), poly(ethylacrylate-methylmethacrylate), poly(methacrylic acid-ethylacrylate), cellulose acetate phthalate, and hydroxypropyl methylcellulose phthalate (HPMCP). The most favorable pH-dependent polymer which is insoluble in acidic pH is poly (methacrylate-methylmethacrylate) with the tradename of Eudragit® S.

When the pH-dependent polymer is insoluble in alkaline pH, the liquid core composition 0 is adjusted to or at an alkaline pH. The preferred pH-dependent polymer that is insoluble in alkaline pH is poly [butyl methacrylate, (2-dimethylaminoethyl) methacrylate, methyl methacrylate], with the trade name of Eudragit® E.

The present invention also provides a method for making the capsule. The method contains the steps of: (1) providing a capsule shell composition which comprises a pH-dependent polymer and optionally a plasticizer; (2) preparing a capsule shell with the capsule shell composition; (3) adjusting a liquid core composition to a pH when the pH-dependent polymer is insoluble; (4) adding the pH-adjusted liquid core composition to the capsule shell; and (5) sealing the capsule shell to form said capsule.

There are three methods to prepare the capsule shell, which are (1) by a plate process, (2) by a rotary die roll process, and (3) by a tubing process. The detail descriptions of the three methods are provided in the “Detailed Description of the Invention” section, infra.

### DETAILED DESCRIPTION OF THE INVENTION

The present invention provides a capsule which contains a capsule shell encapsulating a composition with high liquid content. The capsule shell can be a hard or soft capsule shell, preferably a soft capsule shell. The liquid content in the encapsulated composition can be up to 70% by volume of liquid. The capsule shell is made by a pH-dependent polymer. Optionally, a plasticizer is added to the pH-dependent polymer. In order for the capsule shell to hold the high liquid content, the liquid composition must be adjusted to a pH where the capsule shell polymer is in an insoluble state so that the pH of the composition would not induce the solubilization of the capsule shell. Any conventional methods for adjusting and determining the pH (such as adding inorganic or organic acids/inorganic or organic alkaline to the composition and determine the pH by pH meter) can be used for adjusting the pH of the liquid core composition.

The acid insoluble polymers that can be used for manufacture of the capsule shell include, but are not limited to, (1) a cellulose-based polymer, such as cellulose acetates, cellulose acetate phthalate (CAP), cellulose acetate succinate (CAS), cellulose acetate trimellitate (CAT), hydroxypropyl methylcellulose phthalate (HPMCP), hydroxypropyl methylcellulose acetate succinate (HPMCAS), and carboxymethyl ethyl cellulose (CMEC); (2) starch, sugar, polyols derivatives: starch acetate phthalate, and amylose acetate phthalate; (3) polyvinyl derivatives: polyvinyl alcohol phthalate, polyvinyl acetate phthalate (PVAP), polyvinyl butyrate phthalate, and styrene-maleic acid copolymer; (4) acrylate polymers: polyacrylate, polymethylacrylate, poly(acrylate-methylacrylate), and poly(methacrylate-methylmethacrylate) (Eudragit S). Also, 2-methyl-5-vinyl-pyridine-methyacrylate-methyacrylic acid is insoluble between pH 4 and 7.4. Shellac (which is a resinous excretion of the insect Laccifer (Tachardia) lacca Kerr, order Homoptera, family Coccidae) is also acid insoluble. Zein (which is a prolamin obtainable from corn [molecular weight about 40,000] and which does not contain tryptophan or lysine) is insoluble at pH equal to or less than 11.5.

Alkaline insoluble polymer include, polyvinylacetal diethylamminoacetate, and poly [butyl methacrylate (2-dimethylaminoethyl) methacrylate, methyl methacrylate] (Eudragit E).

Hydroxypropyl methylcellulose (National Formulary XIII) and cellulose acetate phthalate (U.S.P. XVIII) are sometimes referred to as HPMC and CAP, respectively. Hydroxypropyl methylcellulose phthalate is sometimes referred to as HPMCP. At the present time, at least two grades or types of HPMCP are commercially available from the Shinetsu Chemical Company of Tokyo, Japan. These grades or types are known as HP-50 and HP-55. HP-50 has 20-25% methoxyl content, 8-12% hydroxypropoxyl content, and 20-27% carboxybenzoyl content. HP-55 has 18-22% methoxyl content, 6-10% hydroxylpropoxyl content, and 27-35% carboxybenzoyl content. Both HP-50 and HP-55 are soluble in water by the addition of base. HP-50 is dissolved above pH 5. HP-55 is dissolved above pH 5.5.

The preferred polymers or copolymers of acrylate or acrylate derivative include, but are not limited to, polyacrylate, polymethylacrylate, poly(acrylate-methylacrylate), poly(methacrylate-methylmethacrylate), poly(ethylacrylate-methylmethacrylate), poly(ethylacrylate-methylmethacrylate-trimethylammonioethylmethacrylate chloride), and poly(ethylacrylate-methylmethacrylate-trimethylammonioethylmethacrylate chloride). For example, the copolymer of methacrylic acid and methacrylic acid alkyl ester has the following structural unit:

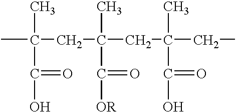

wherein R is a lower alkyl group, in particular, a methyl or ethyl group.

Methacrylic acid/ester copolymer can be prepared according to a number of methods. There are many grades or types of methacrylic acid/ester copolymers that are commercially available. For example, Rohm & Haas Company has the so-called Eudragit® series containing various polymethacrylic acid-methacrylic acid copolymer such as Eudragit®-E, L, S, RL, RS, NE. A list of the Eudragit®-series polymers and their related pH-dependent solubilities are depicted in Table 1.

TABLE 1
Eudragit ®-Polymer Series That Are pH-Dependent

| | | pH of the Liquid Core |
|---|---|---|
| Eudragit ® | Insoluble pH | Composition |
| Eudragit ® L 30D-55 | lower than pH 5.5 | lower than pH 5.5 |
| Eudragit ® L 100 | lower than pH 6.0 | lower than pH 6.0 |
| Eudragit ® L 100-55 | lower than pH 5.5 | lower than pH 5.5 |
| Eudragit ® S 100 | lower than pH 7.0 | lower than pH 7.0 |
| Eudragit ® E 12.5 | higher than pH 5.0 | higher than pH 5.0 |
| Eudragit ® E 100 | higher than pH 5.0 | higher than pH 5.0 |

Thus, based on the pH-dependency of the polymer, the liquid core composition should be adjusted to or at a pH where the polymer is not solubilized. The preferred Eudragit® polymer series to be used in the present invention include Eudragit®)-E, L and S series. Eudragit®-E polymers are polymers or copolymers which are primarily insoluble in alkaline pH (>pH 5.0). Eudragit®-L polymers are primarily insoluble in acid pH (<pH 6.0). Eudragit®-S polymers are primarily insoluble in acidic pH (<pH 7.0).

Specifically, Eudragit L100 contains poly (methacrylic acid, methylmethacrylate) at 1:1 ratio and is sold as solid powder. Eudragit®-S100 contains poly(methacrylic acid, methylmethacrylate) at a 1:2 ratio and is sold as solid powder. Eudragit®-L100-55 contains poly (methacrylic acid, ethylacrylic) at a 1:1 ratio and is sold as solid powder. Eudragit®-E100 contains polyaminomethacrylate-(poly[butylmethacrylate, (2-dimethylaminoethyl)-methacrylate, methylmethacrylate]) at a 2:1 ratio and is sold as solid polymer granules.

Also, due to their mostly insoluble properties in acidic pH, methacrylic acid/ester copolymers are particularly useful for the preparation of drugs for enteric delivery (i.e., for drugs that are intended to be deliver to the duodenal and intestinal regions), because they resist to the acidic pH in the stomach.

Additives, such as dye, glycerin etc. can also be added to the pH-dependent polymers for making the capsule shell.

Optionally, plasticizers can be mixed with the polymers to form the compositions of the capsule shell. Examples of the plasticizers that are suitable to be used with the polymer include (1) polyglycols such as polypropylene glycol, polybutylene glycol and polyethylene glycol (PEG) (400-8000); (2) organic esters such as diethylphthalate (DEP), dibutylphthalate (DBP), dibutyl sebacate (DBS); (3) citrates such as triethyl citrate (TEC), acetyltriethylcitrate (ATEC), acetyltributylcitrate (ATBC), tributylcitrate (TBC), and triacetyl glycerin (triacetin); and (4) oils/glyerides such as castor oil, acetylated monoglyceride, and purified coconut oil.

The preferred plasticizers are polyethylene glycol (e.g., PEG 1000 and 4000), triethyl citrate, tributyl citrate, and triacetin. The preferred amount of plasticizers in the capsule forming compositions is 0%-40% by weight, most favorably 0.01% to 20% by weight.

In addition to the pH-dependent polymer and plasticizer, optionally, the capsule shell can contain gelatin. Also, the inner side of the capsule shell can be deposited with an oily substance to further insulate the liquid core composition from the capsule shell. The oily substance include, but is not limited to, any cominations of vegetable oil, mineral oil, lanolin, emulsified wax, esterified wax, microcrystalline wax, white wax, yellow wax, lubricant lecithin, fatty oil, fatty acid, fish oil, liver oil, whale fatty oil, and polymethyleneglycol.

The liquid core compositions can be any liquid formulations, preferably an herbal decoction or a condensate of the herbal decoction. An herbal decoction is prepared by submerging and boiling an herb or herbal mixture in water to produce a water extract of the herbs. An herbal condensate is generally made by first filtering out the remaining herbs, such as through a sieve (approximately 100 mesh). Then, the filtrate of the decoction is condensed by a decompressed condensation method such as at a temperature of 50 to 60° C. and under vacuum condition (at 30 torr). However, due to large volume of water in the decoction, it is difficult to condense the herbal extract down to less than 20% by volume of water. Therefore, a special capsule shell which can be used to contain the herbal decoction or condensate is particular important for oral delivery of the aqueous herbal extracts.

If the liquid core composition is adjusted to or at a pH lower than 3.0, it is preferred to add an enteric coating layer onto the capsule shell to reinforce the sturdiness of the capsule and to prevent the capsule shell from leakage. Any conventional enteric coating polymers can be used, for example, cellulose acetate phthalate, hydroxypropyl methylcellulose phthalate, polyvinyl acetate phthalate, carboxymethylethylcellulose, co-polymerized methacrylic acid/methacrylic acid methyl esters such as, for instance, Eudragit®L100. The enteric coating can also be applied using water based polymer dispersions, e.g., Aquatic® (FMC Corporation), Eudragit® L 100-55, Coating CE 5142 (BASF). The enteric coating layer can optionally contain a pharmaceutically acceptable plasticizer such as cetanol, triacetin, citric acid esters such as Citroflex® (Pfizer), phthalic acid esters, dibutyl succinate or similar plasticizer. The weight ratio of the polymer to plasticizer should be no less than 10:1, preferably no less than 50:1. Dispersants such as talc, colorants and pigments may also be included into the enteric coating layer.

If the liquid core composition is adjusted to or at a pH greater than 5.0, it is preferred that a film coating is coated onto the capsule shell to ensure the sturdiness of the capsule. The materials for the film-coating layer is chosen among the pharmaceutically acceptable, water soluble polymers such as hydroxypropylmethyl cellulose (HPMC), hydroxypropyl cellulose (HPC), polyvinyl pyrrolidone (PVP), or hydroxymethyl cellulose (HMC). Optionally, a plasticizer can also be added to the film coating layer. The plasticizer which can be used in the film coating layer includes diethyl phthalate, triacetin, and triethyl citrate. In addition, optionally an excipient such as PEG 6000 can be used to replace the plasticizer.

There are at least three ways to prepare the capsule of the present invention: (1) a plate process method; (2) a rotary die process; and (3) a heating method.

(1) The Plate Process

The plate process is especially suitable for making soft capsules. The steps for preparing a capsule using the plate process method are as follows:

(a) A composition for making the capsule shell (“the capsule shell composition”) is spread onto a glass plate or a plastic board.

(b) The capsule shell composition is then dried on the glass plate or plastic board to form a dry capsule shell sheet.

(c) The dried capsule shell sheet is placed on top of a die board containing numeral capsule pockets. The die board is connected to a vacuum.

(d) Vacuum is applied to suck the dried capsule sheet into the capsule pockets of the die board to form a capsule shell.

(e) A liquid core composition is filled into the hollow space of the capsule shell.

(f) A second dried capsule sheet is placed on top of the capsule mold as the cover of the capsule.

(g) The capsule is sealed by applying pressure onto the second dried capsule sheet and extra capsule shell sheet is cut out.

(2) Rotary Die Process

Rotary die process, also known as die roll process, is also particularly suitable for making and filling soft capsules. Detailed description of the process can be found in Lachmann et al., The Theory and Practice of Industrial Pharmacy, 2nd Edition, pages 404-419. The general steps of the process are as follows:

(a) Two capsule shell sheets are supplied to and between a pair of rotating die rolls from the upper side of the dies rolls, one from the right and one from the left. Each roll die has capsule pockets (each pocket is shaped as half of the capsule) in a plurality of row. The two roll dies are close to and confront each other.

(b) A capsule containing two half-capsule shells from the two capsule shell sheets is formed by closing in the rotating dies rolls.

(c) A liquid core composition is injected into the flat capsule with pressure to convert the flat capsule into a swollen one.

(d) The swollen capsule is further sealed by heating.

(3) Tubing Process

The Tubing process is also especially suitable for making soft capsules. The process includes the steps as follows:

(a) The capsule shell composition is supplied to a hollow mold. After drying, the capsule shell composition is stretched out of the mold to form a tubing.

(b) The bottom of the tubing is sealed by heat at temperature as 160° C.

(c) A liquid core composition is filled into the hollow space of the sealed tubing.

(d) The top portion of the tubing is then sealed by heat to form the capsule.

The following examples are illustrative, but not limiting the scope of the present invention. Reasonable variations, such as those occur to reasonable artisan, can be made herein without departing from the scope of the present invention.

In the Examples to be presented below, a popular herbal decoction, Ren-San-Young-Rong-Tang (“Ren-San” is the phonetic words in Chinese for “ginseng”; “Young-Rong” means “better looking”; “Tang” has the same meaning as “decoction”; “Ren-San-Young-Rong-Tong” in its entirety means “a decoction made from ginseng which can make one look nicer”), was used as an example for a liquid core composition. “Ren-San-Young-Rong-Tang” was prepared by adding 20 times by volume of water to the dried ginseng root (cut into pieces) and boiled until about 50% by volume of the water was left in the entire decoction.

### EXAMPLE 1

| (1) | Capsule Shell Composition | |
|---|---|---|
| | (a) | pH-Dependent Polymer |

| | |
|---|---|
| Eudrgit ® E | 20 g |

| | |
|---|---|
| (b) | Plasticizer |

| | |
|---|---|
| Polyethylene Glycol (PEG 1000) | 0.1% |

| | |
|---|---|
| (c) | Procedures for Making the Capsule Shell Composition |

| | |
|---|---|
| 1. | 20 g of Eudragit ® E 20 g was dissolved in 50 ml ethanol. |
| 2. | 0.1% by weight of PEG 1000 was added to the Eudragit ® |

E-solvent solution to form the capsule shell composition.

| | |
|---|---|
| 3. | The capsule shell composition was spread onto a glass plate |

| | and dried at 35-40° C. to form a capsule shell sheet. |
|---|---|
| (2) | Liquid Core Composition |

| | |
|---|---|
| Ren-San-Young-Rong-Tang | pH adjusted to >5.0 |

| (3) | Method for Preparing the Capsule |
|---|---|
| | Plate Process method (see supra). |

### EXAMPLE 2

| (1) | Capsule Shell Composition | |
|---|---|---|
| | (a) | pH-Dependent Polymer |

| | |
|---|---|
| Eudrgit ® B | 20 g |

| | |
|---|---|
| (b) | Plasticizer |

| | |
|---|---|
| Polyethylene Glycol (PEG 1000) | 0.1% |

| | |
|---|---|
| (c) | Procedures for Making the Capsule Shell Composition |

| | |
|---|---|
| 1. | 20 g of Eudragit ® E 20 g was dissolved in 50 ml ethanol. |
| 2. | 0.1% by weight of PEG 1000 was added to the Eudragit ® |

E-solvent solution to form the capsule shell composition.

| | |
|---|---|
| 3. | The capsule shell composition was dried at 35-40° C. |

to form a capsule shell sheet.

| | |
|---|---|
| 4. | Two capsule shell sheets were supplied to and between |

| | a pair of rotating die rolls from the upper side of the die rolls. |
|---|---|
| (2) | Liquid Core Composition |

| | |
|---|---|
| Ren-San-Young-Rong-Tang | pH adjusted to >5.0 |

| | |
|---|---|
| (3) | Method for Preparing the Capsule |

Rotary Die Process method (see supra).

### EXAMPLE 3

| (1) | Capsule Shell Composition | |
|---|---|---|
| | (a) | pH-Dependent Polymer |

| | |
|---|---|
| Eudrgit ® E | 20 g |

| | |
|---|---|
| (b) | Plasticizer |

| | |
|---|---|
| Triacetin | 0.1% |

| | |
|---|---|
| (c) | Procedures for Making the Capsule Shell Composition |

| | |
|---|---|
| 1. | 20 g of Eudragit ® E 20 g was dissolved in 50 ml ethanol. |
| 2. | 0.1% by weight of triacetin was added to the Eudragit ® |

E-solvent solution to form the capsule shell composition.

| | |
|---|---|
| 3. | The capsule shell composition was supplied to a hollow |

mold. After drying, the capsule shell composition was
stretched out of the mold to form a tubing.

| | |
|---|---|
| 4. | The capsule tubing was then subjected to the |

| | “Tubing Process.” |
|---|---|
| (2) | Liquid Core Composition |

| | |
|---|---|
| Ren-San-Young-Rong-Tang | pH adjusted to >5.0 |

| | |
|---|---|
| (3) | Method for Preparing the Capsule |

Tubing Process method (see supra).

### EXAMPLE 4

| (1) | Capsule Shell Composition | |
|---|---|---|
| | (a) | pH-Dependent Polymer |

| | |
|---|---|
| Eudrgit ® S | 20 g |

| | |
|---|---|
| (b) | Plasticizer |

| | |
|---|---|
| Triacetin | 0.1% |

| | |
|---|---|
| (c) | Procedures for Making the Capsule Shell Composition |

| | |
|---|---|
| 1. | 20 g of Eudragit ® S 20 g was dissolved in 50 ml ethanol. |
| 2. | 0.1% by weight of triacetin was added to the Eudragit ® |

S-solvent solution to form the capsule shell composition.

| | |
|---|---|
| 3. | The capsule shell composition was dried at 25-30° C. |

to form a capsule shell sheet.

| | |
|---|---|
| 4. | Two capsule shell sheets were supplied to and between |

| | a pair of rotating die rolls from the upper side of the die rolls. |
|---|---|
| (2) | Liquid Core Composition |

| | |
|---|---|
| Ren-San-Young-Rong-Tang | pH adjusted to <3.0 |

| | |
|---|---|
| (3) | Method for Preparing the Capsule |

Rotary Die Process method (see supra).

### EXAMPLE 5

| (1) | Capsule Shell Composition | |
|---|---|---|
| | (a) | pH-Dependent Polymer |

| | |
|---|---|
| Eudrgit ® S | 20 g |

| | |
|---|---|
| (b) | Plasticizer |

| | |
|---|---|
| Triacetin | 0.1% |

| | |
|---|---|
| (c) | Procedures for Making the Capsule Shell Composition |

| | |
|---|---|
| 1. | 20 g of Eudragit ® S 20 g was dissolved in 50 ml ethanol. |
| 2. | 0.1% by weight of triacetin was added to the Eudragit ® |

E-solvent solution to form the capsule shell composition.

| | |
|---|---|
| 3. | The capsule shell composition was supplied to a hollow |

mold. After drying, the capsule shell composition was stretched out
of the mold to form a tubing.

| | |
|---|---|
| 4. | The capsule tubing was then subjected to the |

| | “Tubing Process.” |
|---|---|
| (2) | Liquid Core Composition |

| | |
|---|---|
| Ren-San-Young-Rong-Tang | pH adjusted to <3.0 |

| | |
|---|---|
| (3) | Method for Preparing the Capsule |

Tubing Process method (see supra).

### EXAMPLE 6

| (1) | Capsule Shell Composition |
|---|---|
| | Gelatin capsules, hard capsules and soft capsules, which were |

commercially available, were used.

| | |
|---|---|
| (2) | Liquid Core Composition |

| | |
|---|---|
| Ren-San-Young-Rong-Tang | No adjustment of pH |

| (3) | Method for Preparing the Capsule | |
|---|---|---|
| | 1. | The inner side of the gelatin capsule was covered with |
| | white wax. | |
| | 2. | The liquid core composition was added to the capsule, |

the capsule was sealed.

Results:

The capsules prepared from EXAMPLES 1-6 were tested for leakage or breakage in room temperature. No leakage or breakage of the capsules was observed, indicating that the capsules made by the above methods have the capacity to hold a liquid core composition with high water content.

While the invention has been described by way of examples and in terms of the preferred embodiments, it is to be understood that the invention is not limited to the disclosed embodiments. On the contrary, it is intended to cover various modifications as would be apparent to those skilled in the art. Therefore, the scope of the appended claims should be accorded the broadest interpretation so as to encompass all such modifications.

We claim:

1. A capsule comprising
a capsule shell comprising a pH-dependent polymer and a plasticizer; wherein the plasticizer is in an amount of 0-40%; and
a liquid core composition which is encapsulated by said capsule shell; wherein said liquid core composition contains up to 70% by volume of liquid; and wherein said pH of said liquid core composition is adjusted to a pH in which the pH-dependent polymer is insoluble.

2. The capsule according to claim 1, wherein said pH-dependent polymer is an acid insoluble polymer.

3. The capsule according to claim 2, wherein said acid insoluble polymer is a cellulose-based polymer which is at least one selected from the group consisting of cellulose acetates, cellulose acetate phthalate, cellulose acetate succinate, cellulose acetate trimellitate, hydroxypropyl methylcellulose phthalate, hydroxypropyl methylcellulose acetate succinate, or carboxymethyl ethyl cellulose.

4. The capsule according to claim 2, wherein said acid insoluble polymer is starch, sugar, or polyols derivative.

5. The capsule according to claim 4, wherein said polyols derivative is at least one selected from the group consisting of starch acetate phthalate or amylose acetate phthalate.

6. The capsule according to claim 2, wherein said pH-dependent polymer is a polyvinyl-based polymer which is at least one selected from the group consisting of polyvinyl alcohol phthalate, polyvinyl acetate phthalate, polyvinyl butyrate phthalate, styrene-maleic acid copolymer, or polyvinylacetal diethylamminoacetate.

7. The capsule according to claim 2, wherein said acid insoluble polymer is a polymer or copolymer of acrylate or acrylate derivative.

8. The capsule according to claim 7, wherein said polymer or copolymer of acrylate or acrylate derivative is at least one selected from the group consisting of polyacrylate, polymethylacrylate, poly(acrylate-methylacrylate), poly(methacrylate-methylmethacrylate), poly(ethylacrylate-methylmethacrylate), poly(ethylacrylate-methylmethacrylate-trimethylammonioethylmethacrylate), poly(ethylacrylate-methylmethacrylate-trimethylammonioethylmethacrylate chloride), or 2-methyl-5-vinyl-pyridine-methyacrylate-methyacrylic acid.

9. The capsule according to claim 2, wherein said polymer is zein or shellac.

10. The capsule according to claim 1, wherein said pH-dependent polymer is an alkaline insoluble polymer.

11. The capsule according to claim 10, wherein said alkaline insoluble polymer is poly[butyl methacrylate, (2-dimethylaminoethyl) methacrylate, methyl methacrylate] or polyvinylacetal diethylamminoacetate.

12. The capsule according to claim 1, wherein the plasticizer is selected from the group consisting of at least one selected from the group consisting of glycerin, propylene glycol, polyethylene glycol (PEG 200-6000), diethyl phthalate, dibutyl phthalate, dibutyl sebacate, triethyl citrate, acetyltriethyl citrate, acetyltributyl citrate, tributyl citrate, triacetyl glycerin, castor oil, acetylated monoglyceride, and coconut oil.

13. The capsule according to claim 1, wherein the plasticizer is in an amount of 0.01-10%.

14. The capsule according to claim 1, wherein said capsule shell further comprises gelatin.

15. The capsule according to claim 1, wherein an oily substance is added to an inner side of said capsule shell.

16. The capsule according to claim 15, wherein said oily substance is at least one selected from the group consisting of vegetable oil, mineral oil, lanolin, emulsified wax, esterified wax, microcrystalline wax, white wax, yellow wax, lubricant lecithin, fatty oil, fatty acid, fish oil, liver oil, whale fatty oil, and polymethyleneglycol.

17. The capsule according to claim 1, wherein said capsule shell is a soft capsule shell.

18. The capsule according to claim 1, wherein said liquid core composition comprises up to 50% by volume of liquid.

19. The capsule according to claim 1, wherein said liquid is water.

20. The capsule according to claim 1, wherein said liquid core composition is an herbal pharmaceutical composition.

21. The capsule according to claim 1, wherein said liquid core composition is adjusted to or at an acidic pH when said pH-dependent polymer is an acid insoluble polymer.

22. The capsule according to claim 1, wherein said liquid core composition is adjusted to or at an alkaline pH when said pH-dependent polymer is an alkaline insoluble polymer.

23. A method for making the capsule according to claim 1 comprising:
providing a capsule shell composition comprising said pH-dependent polymer and optionally said plasticizer;
preparing said capsule shell with said capsule shell composition;
adjusting said liquid core composition to a pH when said pH-dependent polymer is insoluble;
adding said pH-adjusted liquid core composition to said capsule shell; and
sealing said capsule shell to form said capsule.

24. The method according to claim 23, wherein said capsule shell is prepared by a plate process.

25. The method according to claim 23, wherein said capsule shell is prepared by a rotary die process.

26. The method according to claim 23, wherein said capsule shell is prepared by a tubing process.

27. The method according to claim 21, further comprising coating an oily substance at an inner side of said capsule shell.

28. The method according to claim 27, wherein said oily substance is at least one selected from the group consisting of vegetable oil, mineral oil, lanolin, emulsified wax, esterified wax, microcrystalline wax, white wax, yellow wax, lubricant lecithin, fatty oil, fatty acid, fish oil, liver oil, whale fatty oil, and polymethyleneglycol.
